(19) United States
(12) Patent Application Publication
(10) Pub. No.: US 2014/0190473 A1
(43) Pub. Date: Jul. 10, 2014

(54) INHALATION PEN

(71) Applicant: Hans Haindl, Wennigsen (DE)

(72) Inventor: Hans Haindl, Wennigsen (DE)

(21) Appl. No.: 14/119,010

(22) PCT Filed: May 18, 2012
(86) PCT No.: PCT/EP2012/059283

§ 371 Date: Mar. 12, 2014

(30) Foreign Application Priority Data
May 20, 2011 (DE) 20 2011 101 012.4

(51) Int. Cl. A61M 11/08; A61M 11/04; A61M 15/00

(52) U.S. Cl. CPC A61M 11/08; A61M 15/009; A61M 11/04; A61M 15/0008; A61M 15/0051
USPC 128/200.23; 128/203.12; 128/200.14; 128/203.15; 128/203.21

(57) ABSTRACT

The present invention relates to an inhaler for administering a defined dose of an active ingredient into the respiratory tracts of a user. The inhaler has an elongate shape defining a longitudinal axis and a cross-sectional profile, wherein the maximum cross-sectional area is at most 4 cm² and the maximum extension of the cross-section is at most 2.85 cm.

The present invention relates to an inhaler for administering a defined dose of an active ingredient, in particular a drug, into the respiratory tracts of a user.

A lot of inhalation devices are used for the treatment of various respiratory diseases, but partially also for the intake of drugs for other purposes. The following inhalation devices are to be mentioned in this connection: the so-called metered dose inhalers (MDI), which upon compression release a defined amount of a liquid drug from a cartridge pressurized by a propellant, as well as the so-called powder inhalers, in which a dose of powder is located in a gelatin or plastic capsule, opened in the inhaler and the powder is swirled up by the user's breathing air stream and inhaled in a finely distributed way. Other inhalers are based, for example, on atomizing a drug in micronozzles.

All these systems work well to a greater or lesser extent but are disadvantageous to a different extent in that they are relatively bulky apparatuses which not necessarily can be worn inconspicuously on the body, for example, when wearing light summer clothes or doing sports. Since the respective patients as a rule, however, are dependant on having the required drugs with them at any time, it is desirable to provide an inhaler which is configured such that it can be worn on the body without any adverse effect.

The present invention is based, i.a., on the idea of configuring a fully functioning inhaler in the form of a ballpoint pen which preferably contains several individual drug doses and/or a daily dose. In other words, the inhaler according to the invention should have an elongate, relatively slim shape which preferably permits wearing the inhaler, for example, in one's shirt or jacket pocket. To this end, the inhaler can preferably comprise a clip which is adapted for fixing the inhaler in a shirt or jacket pocket, as is typical for ballpoint pens. It is particularly preferred that the entire appearance of the inhaler is similar to a ballpoint pen and can at first glance optically not be distinguished from such a ballpoint pen. The inhaler can comprise, for example, a push-button for releasing a drug dose, wherein said push-button is similar to the usual ballpoint pen push-buttons.

Accordingly, the present invention relates to an inhaler for the, preferably repeated, administration of a defined dose of an active ingredient into the respiratory tracts of a user. The inhaler has an elongate shape defining a longitudinal axis and a cross-sectional profile, wherein the maximum cross-sectional area is at most 4 cm² and the maximum linear extension of the cross-section is at most 2.85 cm.

The cross-sectional profile at different axial positions is defined by respective sections perpendicular to the longitudinal axis. The maximum cross-sectional area results from the maximum of the thus defined cross-sectional areas at all axial positions. Likewise it is possible to determine a maximum extension in any cross-section, e.g. the diameter in the case of a circle or the diagonal in the case of a rectangle. The maximum extension of all cross-sections perpendicular to the longitudinal axis should accordingly be at most 2.85 cm. In other words, the greatest cross-section in an extreme case should not deviate from a circle to a greater extent than a square. As a rule, the cross-section should relate to that of the elongate shape as such and not embrace possible additionally mounted components, such as, e.g., a clip for fixing.

According to a preferred embodiment of the inhaler according to the invention, the maximum cross-sectional area is at most 3 cm², preferably at most 2 cm², particularly preferably at most 1.5 cm². According to a preferred embodiment of the inhaler according to the invention, the maximum extension of the cross-section is at most 2.2 cm, preferably at most 1.5 cm, particularly preferably at most 1.1 cm.

According to a preferred embodiment of the inhaler according to the invention, it has essentially the shape of a ballpoint pen. The cross-section of the inhaler can be, for example, circular, elliptical, square, rectangular, pentagonal, hexagonal or polygonal. The profile or the cross-sectional area can vary along the longitudinal axis and can be, for example, convex, concave, conical or the like. The length of the inhaler is preferably between 8 cm and 18 cm, more preferably between 10 cm and 16 cm, particularly preferably between 12 cm and 15 cm.

According to a preferred embodiment of the inhaler according to the invention, the inhaler comprises a clip which is preferably adapted for fixing the inhaler in a shirt and/or jacket pocket.

According to a preferred embodiment, a first end of the inhaler comprises a suction orifice. Said suction orifice preferably has an orifice area between 10 mm² and 40 mm², particularly preferably between 15 mm² and 25 mm². The suction orifice is preferably substantially circular and has an orifice diameter between 3 mm and 7 mm, particularly preferably between 4 mm and 6 mm.

Preferably, the first end is movable such that it can be moved from a first position, in which the first end is aligned substantially parallel to the longitudinal axis of the inhaler, into a second position, in which the first end and the longitudinal axis of the inhaler include an angle of at least 45°, preferably at least 60°, more preferably at least 75°, particularly preferably about 90°. The first end is preferably tiltable and/or rotatable relative to the longitudinal axis of the inhaler. Alternatively or additionally, the first end can be flexible and/or bendable for this purpose. It is thus possible that the inhaler essentially remains in an approximately perpendicular orientation irrespective of whether it is used by the user in an upright, sitting or recumbent position.

The active ingredient can be basically in the form of a liquid, preferably a volatile liquid, or in the form of a powder. The active ingredient preferably contains one or more drugs.

According to a preferred embodiment, the inhaler comprises a mixing means which is adapted for swirling an air stream within the inhaler. The air stream preferably is formed in that the user sucks air through the suction orifice. To this end, the inhaler preferably comprises one or more orifices for admitting air into the inhaler. The air sucked-in drags the active ingredient with it, said active ingredient being distributed as finely as possible by the swirling motion.

According to a preferred embodiment, the inhaler comprises an inspection window through which the number of available doses can be optically checked. Said inspection window can be arranged and dimensioned such that it can be recognized whether there is still a predetermined minimum amount of doses (e.g., at least three, at least five or at least ten doses) in the inhaler.

According to a preferred embodiment of the inhaler according to the invention, said inhaler comprises a release mechanism, wherein the actuation of said release mechanism causes the provision of a defined dose of the active ingredient. Depending on the type of the inhaler and/or the active ingredient, the provision can comprise the feature of metering and/or dispensing a defined amount of liquid or the feature of conveying and/or dispensing a previously defined dose of a liquid or powder.

It is preferred that the release mechanism comprises a push-button that is preferably attached at the end of the inhaler which is opposite the first end. Preferably, the push-button is configured such that its appearance corresponds to that of push-buttons of conventional ballpoint pens. The one-time actuation of the push-button entails the release and/or provision of a dose of the active ingredient.

According to a preferred embodiment of the inhaler according to the invention, said inhaler comprises several portions of the defined dose of the active ingredient, wherein said portions are separated from each other. These portions can be individually packaged and/or sealed. A liquid active ingredient can be provided, for example, in individual capsules. In this case, the actuation of the release mechanism preferably entails that one portion of the defined dose of the active ingredient is conveyed to a predetermined position and/or opened.

According to a preferred embodiment of the inhaler according to the invention, the portions are arranged in dose containers on a conveyor belt, preferably in bubbles or blisters of a blister strip. The actuation of the release mechanism preferably entails that the conveyor belt or blister strip is moved forward by a distance between adjacent portions and/or that a dose container of the conveyor belt or a bubble of the blister strip is opened. To this end, the inhaler preferably comprises a cannula tip, a mandrel, a knife or the like. The actuation of the release mechanism preferably entails that a dose container of the conveyor belt or a bubble of the blister strip is moved into the cannula tip, the mandrel or the knife such that it is cut open or punctured. The active ingredient in the dose container or bubble is thus released and can be introduced into the respiratory tracts of the user by sucking in air. In the case of an active ingredient in powder form, the bubble is preferably cut such that the active ingredient can drop or trickle out of the bubble. When the bubble contains a liquid active ingredient, said active ingredient is preferably volatile so that its rapid evaporation is caused by opening or puncturing the bubble. The evaporated active ingredient can then be simply inhaled when sucking in air.

According to a preferred embodiment of the inhaler according to the invention, the release mechanism, i.e. for example the push-button, engages by means of a hook or by means of teeth with pawl-like projections on or orifices in the conveyor belt or blister strip. In this way, the release mechanism moves the conveyor belt forward by one position and thus conveys the next filled bubble to or into the knife or the mandrel. Subsequently, the hook is removed in order to be able to engage with the next projection. This is done, for example, by pushing and releasing a spring-loaded push-button. The release mechanism can also be realized by means of rotating a, preferably spring-loaded, rotary knob or the like.

According to a preferred embodiment of the inhaler according to the invention, said inhaler comprises a flexible seat area which is arranged such that a portion of the defined dose of the active ingredient can be placed or kept on it. A powder portion, for example, drops or trickles out of a cut open bubble onto said seat area upon actuation of the release mechanism. The user's subsequent sucking on the suction orifice then preferably entails that the flexible seat area is deformed to such an extent that the portion placed on it is released. The active ingredient is thus prevented from dropping out of the suction orifice before the user duly sucks on it upon cutting open the bubble.

The principle according to the invention can also be applied to liquid inhalers. To this end, the shape of the pressurized cartridges is preferably modified such that they are longer and thinner than conventional pressurized cartridges. Furthermore, the principle according to the invention can also be applied to powder inhalers, in which the defined dose of the active ingredient is scraped in the form of a defined amount of powder from a block of active ingredient by means of a scraping mechanism and thus provided and administered.

The inhaler according to the invention is advantageous, i.a., because it is, on the one hand, extremely small and, on the other hand, configured such that it can be permanently worn on the body, for example in a shirt or jacket pocket, without any adverse effects. Thus, the user does not have to carry along an inhaler having perhaps a bulky shape and size in a sufficiently large bag. Moreover, the shape of a ballpoint-pen permits the user to easily hide that he/she needs permanent medication. Though having a small and simple shape, the inhaler according to the invention enables the inhalation doses for one day, preferably for one week and particularly preferably for a month to be carried along.

In addition to the aforementioned advantages, which are essentially based on the dimensioning and shape of the inhaler according to the invention, further advantages over the prior art are made possible by the features of the preferred embodiments. The inhaler according to the invention ensures, for example, an extremely simple while at the same time reliable application. Moreover, the inhaler according to the invention can be manufactured in a simple and cost-efficient way.

Preferred embodiments of the inhaler according to the invention are described in the following with reference to the following Figures in more detail:

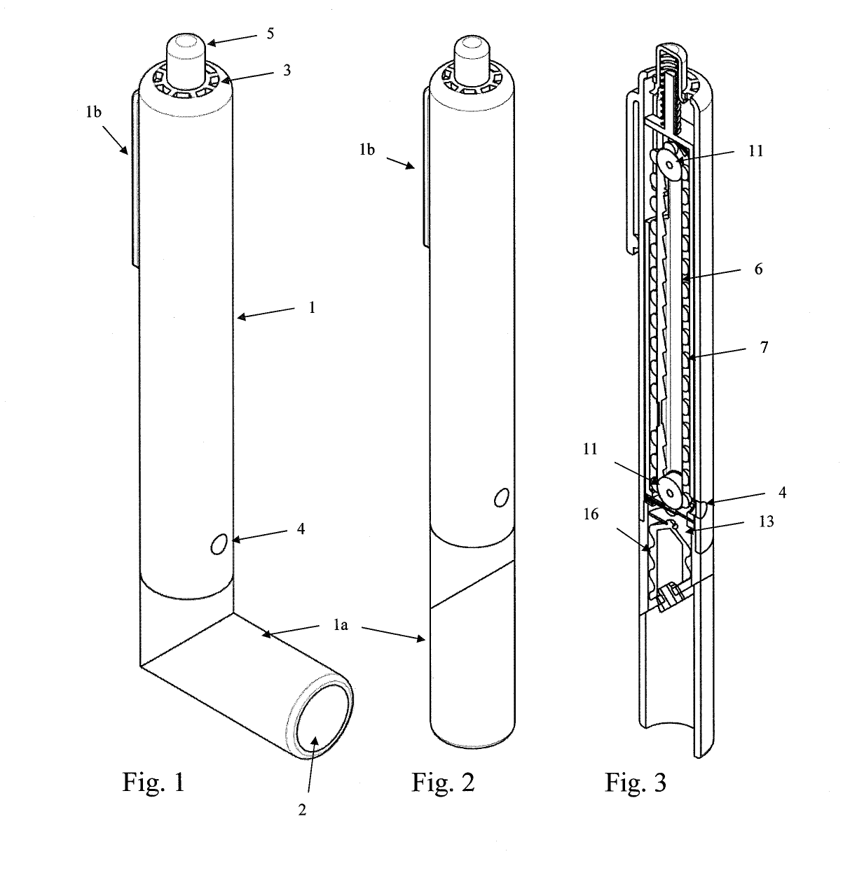

FIGS. 1 and 2 show perspective views of a preferred embodiment of the inhaler according to the invention;

FIG. 3 shows a perspective sectional view of the inhaler according to FIG. 2;

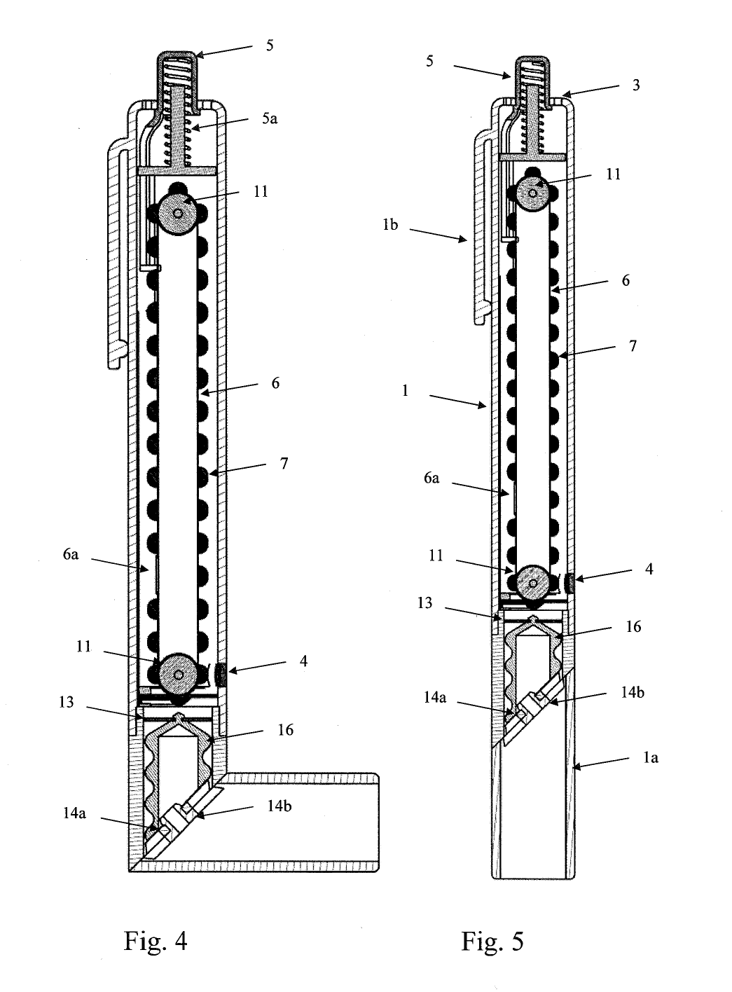

FIGS. 4 and 5 show sectional views of the inhaler according to FIGS. 1 and 2;

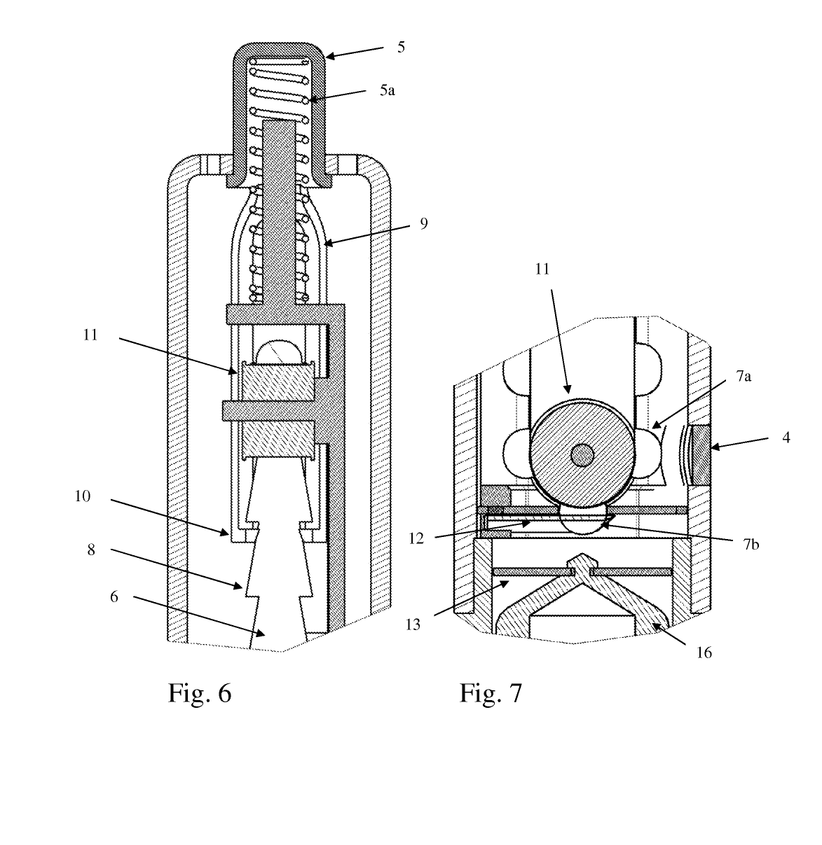

FIGS. 6 and 7 show sectional views of details of the inhaler according to FIGS. 1 and 2;

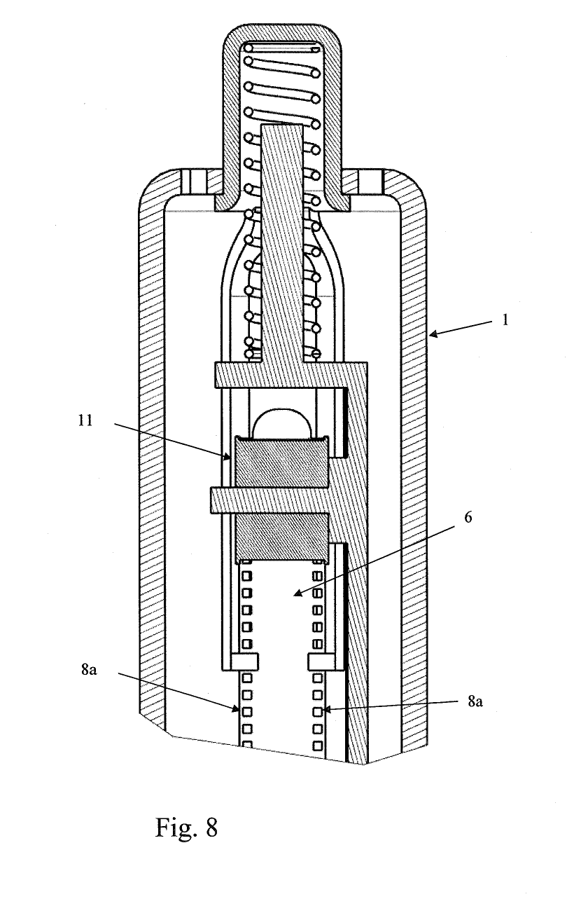

FIG. 8 shows a sectional view of a preferred embodiment of the inhaler according to the invention;

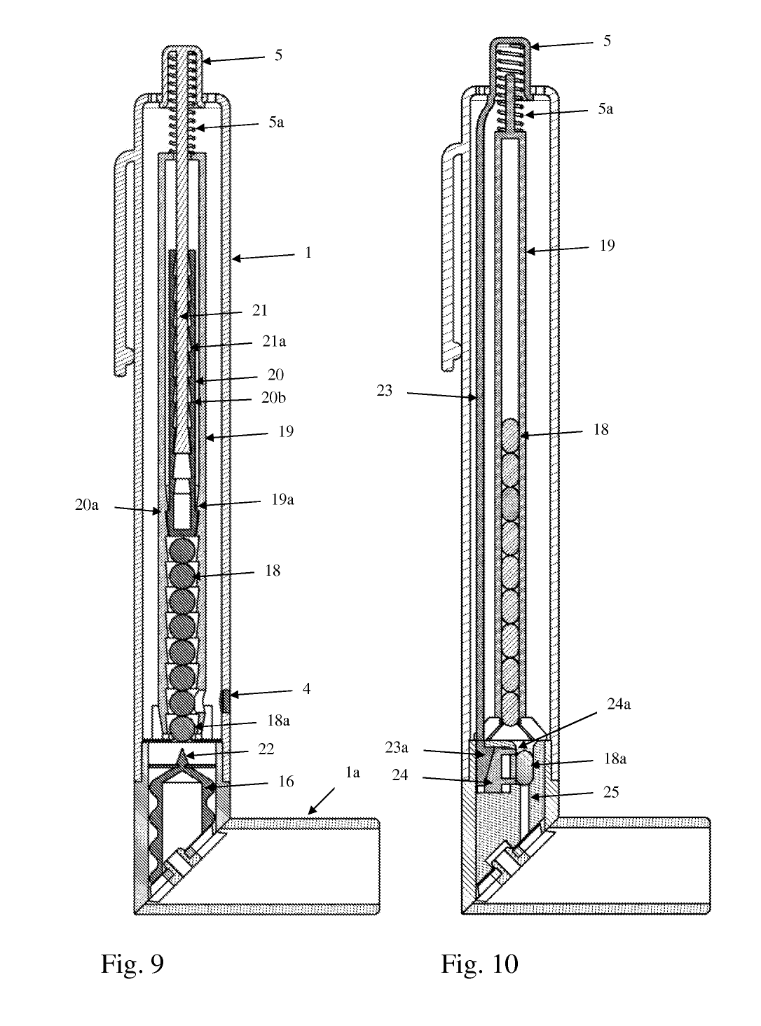

FIG. 9 shows a sectional view of a further preferred embodiment of the inhaler according to the invention;

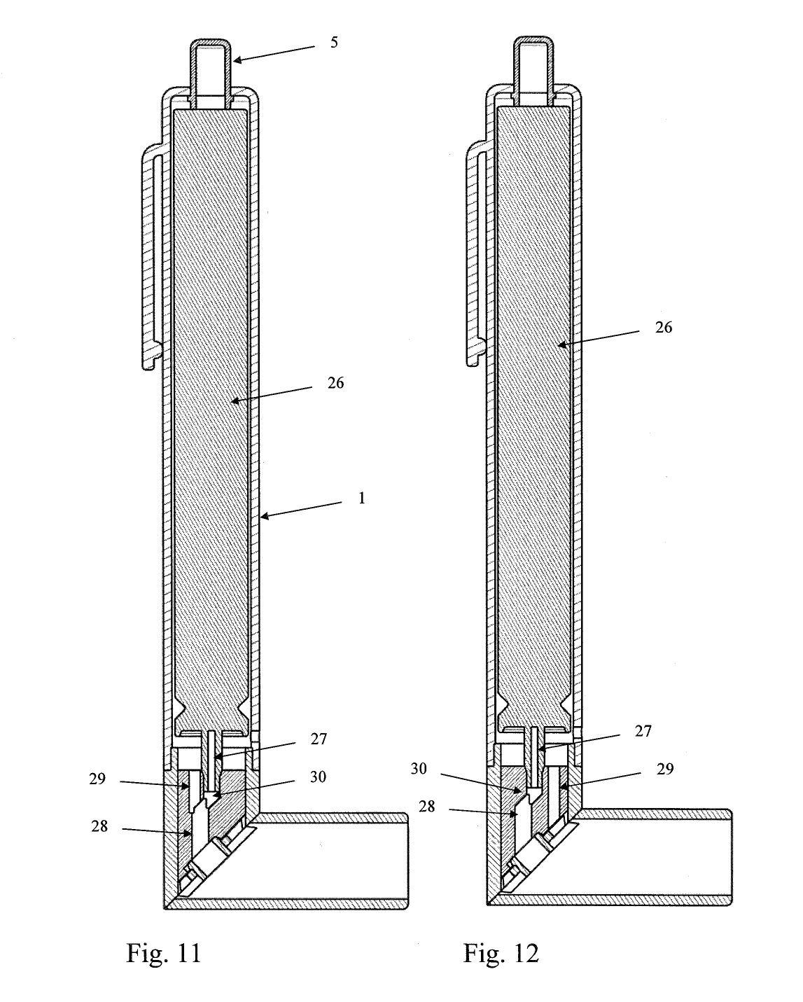

FIG. 10 shows a sectional view of a further preferred embodiment of the inhaler according to the invention;

FIG. 11 shows a sectional view of a further preferred embodiment of the inhaler according to the invention;

FIG. 12 shows a sectional view of a further preferred embodiment of the inhaler according to the invention;

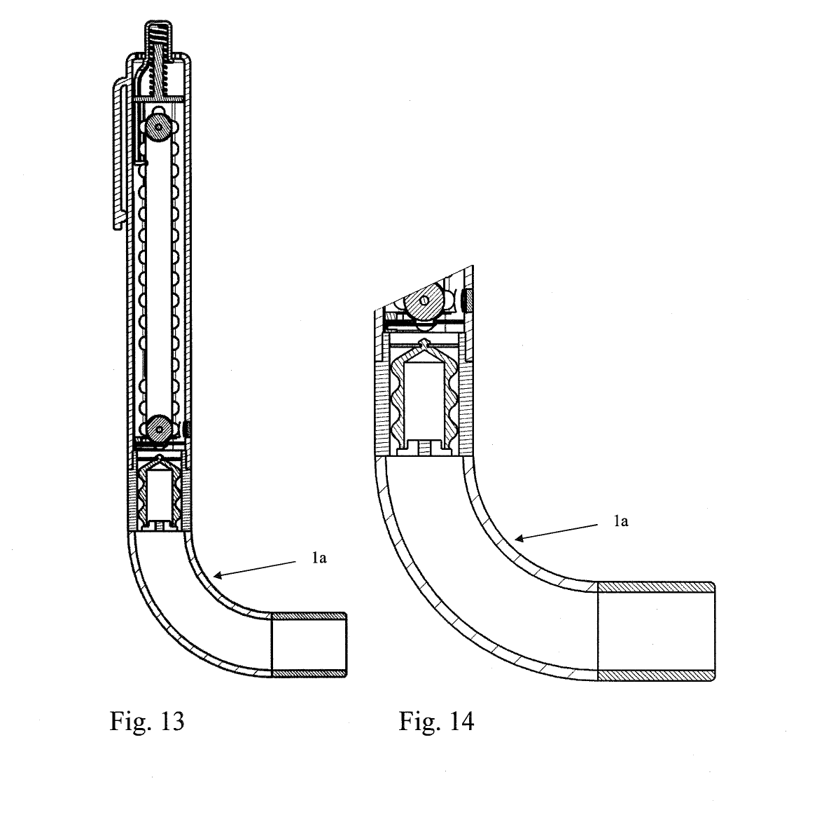

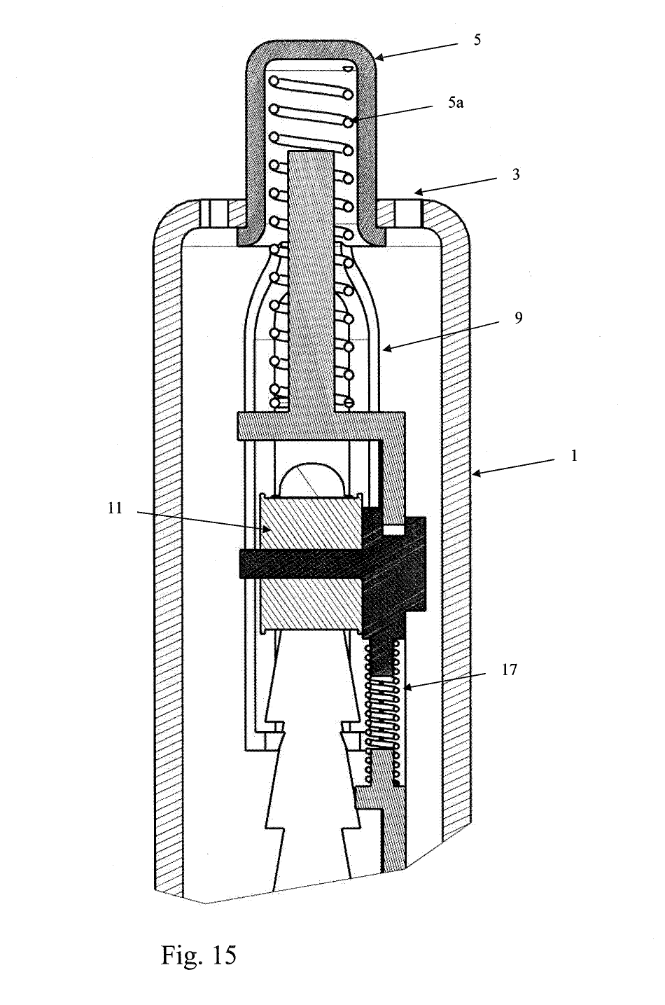

FIGS. 13 and 14 show sectional views of further preferred embodiments of the inhaler according to the invention;

FIG. 15 shows a sectional view of a further preferred embodiment of the inhaler according to the invention.

FIGS. 1-7 show different views of a first preferred embodiment of the inhaler according to the invention. This inhaler is an inhalation apparatus which is actuated by breathing air and has an elongate, approximately cylindrical shape. The inhaler according to the preferred embodiment has an substantially cylindrical housing 1 comprising a suction orifice 2 and one or more additional air orifices 3. The suction orifice 2 is provided on a first, lower end 1a of the cylindrical housing 1, wherein said lower end 1a can be moved from a first position, which is shown in FIG. 2 and in which the first end 1a is aligned substantially parallel to the longitudinal axis of the inhaler, into a second position, which is shown in FIG. 1 and in which the first end 1a and the longitudinal axis of the inhaler include an angle of about 90°.

In the illustrated embodiment, this movement is performed by rotating the first end relative to the remaining housing of the inhaler. There is a conveyor belt or blister strip 6 inside the housing 1 comprising several chambers or bubbles 7 each containing a dose of an active ingredient or a drug. In the distance between adjacent bubbles 7, the blister strip comprises pawl-like projections 8 which can be engaged with a traction member 9 by means of hooks 10 in order to move forward the blister strip 6 in the housing 1. In this embodiment, the blister strip 6 is connected to form an endless loop and guided on guide rolls 11, one of which is located in the upper region of the housing 1 and the other of which is located in the lower region of the housing 1.

The traction member 9 communicates with a push-button 5 which is held by a spring 5a in its initial position as shown in FIG. 6. When a user pushes the push-button 5 from the outside, the preferably flexible or bendable traction member 9 passes with its hooks 10 along the corresponding projections 8 of the blister film 6 until the hooks 10 engage with and/or latch into the notches formed by the projections 8. When the push-button 5 is released by the user, the traction member 9 is moved upwards by the integrated spring 5a, wherein the traction member 9 moves the blister strip 6 forward by the distance between two adjacent bubbles by means of the hooks 10.

The bubble or blister 7 at the right bottom of the blister strip is thus moved from the position 7a into the position 7b (cf. FIG. 7), in which the bubble or blister 7 is cut open by a knife 12. The blister 7 is thereby opened and its content drops onto a flexible membrane 13 held on a ring member. The elastic membrane 13 seals the area above the membrane against the housing wall downwards so that the dropped drug dose cannot escape from the inhaler via the suction orifice 2. A corresponding membrane can also be arranged further above in order to prevent the drug or the active ingredient from falling out of the inhaler through additional air orifices 3 when the inhaler is tilted or shaken.

When the user sucks on the suction orifice 2 with his/her mouth, air is drawn into the inside of the housing 1 through the additional air orifices 3. This entails that the outer edges of the membrane 13 turn down so that the drug particles located on the membrane 13 are dragged with the air stream and carried along into the respiratory tracts of the user. The only schematically depicted structure 16 forms a swirl chamber that swirls the air flowing through it such that the active ingredient or drug is mixed with the air to a sufficiently fine extent.

The housing 1 further contains an inspection window 4 through which the user can examine the blister 7 in the position 7a for whether it is still filled. The individual blisters are preferably provided with numbers which indicate to the user in the inspection window 7 how many doses of the active ingredient are left. Alternatively, an inspection window 4 can be provided in the form of an elongate slit by means of which the entire row of blisters can be examined for faultlessness.

Finally, the inhaler according to the preferred embodiment comprises a clip or clamp 1b which is adapted for fixing the inhaler in a shirt or jacket pocket.

It goes without saying that the structural and dimensional details illustrated in FIGS. 1-7 are only to be understood as examples. The shape of the housing 1, for example, is not restricted to a cylinder and can be configured, for example, convexly, concavely or conically. The cross-section of the housing 1 can deviate from the illustrated circular shape and be, for example, elliptical, rectangular, square, pentagonal, hexagonal or polygonal. The traction element 9 can also be differently configured in an alternative embodiment and comprise, for example, only one hook 10 on one side of the conveyor belt or blister strip 6. Accordingly, the blister strip can also be configured such that the illustrated pawl-like projections 8 are only arranged on one side of the blister strip. Alternatively, the blister strip can also be configured so as to comprise a row of orifices 8a as depicted in the embodiment according to FIG. 8, wherein hooks or peaks of the traction element 9 can engage with said orifices. Said orifices 8a additionally or alternatively can serve the purpose of stabilizing the blister strip or the conveyor belt on the guide rolls 11 which are preferably equipped with toothed rings to this end.

In the embodiment according to FIGS. 1-7, the first end 1a of the inhaler can be moved from the first position into the second position in that the first end 1a of the inhaler is rotated relative to the longitudinal axis of the inhaler. To this end, as apparent from FIGS. 4 and 5, the inhaler is preferably configured with a hub 14a and an axle 14b, which engage with each other such that the aforementioned rotary movement is made possible. According to a further preferred embodiment which is illustrated in FIGS. 13 and 14, the first end 1a of the inhaler is configured flexibly such that it can be bent relative to the longitudinal axis of the inhaler by at least 45°, preferably at least preferably at least 60°, more preferably at least 75° and particularly preferably about 90°.

In order to configure the blister strip 6 with sufficient elastic tension that the blister strip remains tightened between the two guide rolls 11, the blister strip 6 according to the embodiment of FIGS. 1-7 is provided with an elastic member 6a that keeps the blister strip 6 tight. According to a further preferred embodiment which is shown in FIG. 15, a spring 17 can be provided in addition or as an alternative to the elastic member 6a, wherein one of the two guide rolls 11 is resiliently supported by said spring 17. It is also possible to resiliently support both of the guide rolls 11 by means of springs 17.

A further preferred embodiment of the inhaler according to the invention is depicted in FIG. 9. Instead of a blister strip 6 comprising individual blisters 7, the inhaler according to this embodiment comprises a series of active-ingredient capsules 8 arranged on top of each other in a tube 19. A movable cylinder 20 is located within said tube 19, said cylinder 20 being provided with a projection 20a engaging with the pawl-like projections 19a within the tube 19 such that the cylinder 20 can be moved only downwards within the tube 19. A piston 21 is movably supported in the cylinder 20, one end of said piston 21 likewise comprising projections 21a which engage with pawl-like projections 20b on the inside of the cylinder 20 such that the piston 21 can be moved only upwards within the cylinder 20. This combination of two ratchets engaging with each other entails that the piston 21 is pushed downwards along with the cylinder 20 when a user pushes the push-button 5, whereas only the piston is drawn upwards on account of the effect of the spring 5a, when the push-button 5 is released.

On account of the pressure exerted by the cylinder 20 on the series of capsules 18, the lowermost capsule 18a is driven into the mandrel 22 whereby it is opened. By means of an inspection window it can be verified whether there are still capsules 18 left within the tube 19.

A further preferred embodiment of the inhaler according to the invention is depicted in FIG. 10. This inhaler also comprises a tube 19 within which a series of capsules 18 are arranged. The lowermost capsule is pushed into or maintained in the position 18a by gravity. In this embodiment, the push-button 5 is connected to a wedge-shaped member 23a via a rod system 23. Said wedge-shaped member 23a interacts with a likewise wedge-shaped cutting member 24 that comprises two mandrels or knives 24a. When a user pushes the push-button 5, the wedge-shaped member 23a is pushed downwards by means of the rod system 23, whereby the likewise wedge-shaped cutting member 24 is moved to the right so that the capsule 18a is punctured or cut open by the two mandrels or knives. The active ingredient contained in the capsule can then escape via the bore 25 to the suction orifice 2 at the first end 1a of the inhaler.

Finally, two further preferred embodiments of the inhaler according to the invention are illustrated in FIGS. 11 and 12, said embodiments being based on the principle of known liquid inhalers. In these embodiments the active ingredient is provided in the form of a liquid in an active ingredient reservoir 26. The inhaler can comprise a pump to provide a defined amount of liquid or the active ingredient reservoir can be pressurized by a propellant. When the push-button 5 is pushed, the reservoir 26 along with the tube 27 is pushed against the limit stop or edge 30 so that a non-depicted valve is opened and fluid can escape through the tubes 27 and 28 and thus reaches the suction orifice 2. Preferably, a further tube 29 is provided which either in combination with tube 28 (FIG. 11) or alone (FIG. 12) enables pressure compensation below the reservoir 26.

1-25. (canceled)

26. An inhaler for repeatedly administering a defined dose of an active ingredient into the respiratory tracts of a user, wherein the inhaler has an elongate shape defining a longitudinal axis and a cross-sectional profile, wherein the maximum cross-sectional area is at most 4 cm² and the maximum linear extension of the cross-section is at most 2.85 cm.

27. The inhaler according to claim 26, wherein the maximum cross-sectional area is at most 3 cm².

28. The inhaler according to claim 26, wherein the maximum cross-sectional area is at most 2 cm².

29. The inhaler according to claim 26, wherein the maximum cross-sectional area is at most 1.5 cm².

30. The inhaler according to claim 26, wherein the maximum extension of the cross-section is at most 2.2 cm.

31. The inhaler according to claim 26, wherein the maximum extension of the cross-section is at most 1.5.

32. The inhaler according to claim 26, wherein the maximum extension of the cross-section is at most 1.1 cm.

33. The inhaler according to claim 26, wherein the inhaler has essentially the shape of a ballpoint pen.

34. The inhaler according to claim 26, wherein the inhaler comprises a clip which is adapted for fixing the inhaler in a shirt and/or jacket pocket.

35. The inhaler according to claim 26, wherein a first end of the inhaler comprises a suction orifice.

36. The inhaler according to claim 35, wherein the first end is movable such that it can be moved from a first position, in which the first end is aligned substantially parallel to the longitudinal axis of the inhaler, into a second position, in which the first end and the longitudinal axis of the inhaler include an angle of at least 45°.

37. The inhaler according to claim 35, wherein the first end is movable such that it can be moved from a first position, in which the first end is aligned substantially parallel to the longitudinal axis of the inhaler, into a second position, in which the first end and the longitudinal axis of the inhaler include an angle of at least 60°.

38. The inhaler according to claim 35, wherein the first end is movable such that it can be moved from a first position, in which the first end is aligned substantially parallel to the longitudinal axis of the inhaler, into a second position, in which the first end and the longitudinal axis of the inhaler include an angle of at least 75°.

39. The inhaler according to claim 35, wherein the first end is movable such that it can be moved from a first position, in which the first end is aligned substantially parallel to the longitudinal axis of the inhaler, into a second position, in which the first end and the longitudinal axis of the inhaler include an angle of at least about 90°.

40. The inhaler according to claim 35, wherein the first end is tiltable and/or bendable relative to the longitudinal axis of the inhaler.

41. The inhaler according to claim 35, wherein the first end is rotatable relative to the longitudinal axis of the inhaler.

42. The inhaler according to claim 26, wherein the active ingredient is in the form of a liquid.

43. The inhaler according to claim 26, wherein the active ingredient is in the form of a volatile liquid.

44. The inhaler according to claim 26, wherein the active ingredient is in the form of a powder.

45. The inhaler according to claim 26, wherein the inhaler comprises a mixing means which is adapted for swirling an air stream within the inhaler.

46. The inhaler according to claim 26, wherein the inhaler comprises an inspection window through which the number of available doses can be optically checked.

47. The inhaler according to claim 26, wherein the inhaler comprises a release mechanism and wherein the actuation of said release mechanism causes the provision of a defined dose of the active ingredient.

48. The inhaler according to claim 47, wherein the release mechanism comprises a push-button.

49. The inhaler according to claim 26, wherein the inhaler comprises several portions of the defined dose of the active ingredient, wherein said portions are separated from each other.

50. The inhaler according to claim 49, wherein the portions are individually packaged and/or sealed.

51. The inhaler according to claim 47, wherein the actuation of the release mechanism entails that one portion of the defined dose of the active ingredient is conveyed to a predetermined position and/or opened.

52. The inhaler according to claim 49, wherein the portions are arranged in dose containers on a conveyor belt, preferably in bubbles of a blister strip.

53. The inhaler according to claim 52, wherein the actuation of the release mechanism entails that the conveyor belt or blister strip is moved forward by a distance between adjacent portions.

54. The inhaler according to claim 52, wherein the actuation of the release mechanism entails that a dose container of the conveyor belt or a bubble of the blister strip is opened.

55. The inhaler according to claim 47, wherein the inhaler comprises a cannula tip or a knife and wherein the actuation of the release mechanism entails that a dose container of the conveyor belt or a bubble of the blister strip is moved into the cannula tip or the knife such that it is cut open.

56. The inhaler according to claim 52, wherein the release mechanism comprises a push-button and wherein said push-button engages by means of a hook with pawl-like projections on the conveyor belt or blister strip.

57. The inhaler according to claim 26, wherein the inhaler comprises a flexible seat area which is arranged such that a portion of the defined dose of the active ingredient can be placed on it.

58. The inhaler according to claim 32, wherein the inhaler comprises a suction orifice and wherein sucking on the suction orifice entails that the flexible seat area is deformed to such an extent that the portion placed on it is released.
